(19) United States
(12) Patent Application Publication
(10) Pub. No.: US 2007/0208366 A1
(43) Pub. Date: Sep. 6, 2007

(54) Minimally invasive tissue expander systems and methods

(76) Inventors: Richard Pellegrino, Upton, MA (US); Sean Selover, Westport, MA (US); Sara Dziedzic, Braintree, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/368,022

(22) Filed: Mar. 3, 2006

(51) Int. Cl. A61M 29/00

(52) U.S. Cl. 606/198

(57) ABSTRACT

A tissue expander system for expanding tissue, skin and muscle along a pathway to a surgical site through a minimally invasive incision includes a tissue expander having a plurality of arms adapted to bear against tissue, the plurality of arms coupled at a first end to an actuating member and at a second end to a shaft, movement of the actuating member relative to the shaft adjusts the arms between a first position having a first diameter and a second position having second diameter greater than the first diameter and an outer sleeve having a lumen sized and shaped to receive the tissue expander in the second position.

### BACKGROUND

In minimally invasive surgical procedures providing access to the surgical site is a balancing act between minimizing the size of the incision and providing enough room for the surgeon to manipulate the instruments to perform the surgery. Access devices, including expandable tubular retractors and ports are used to retract the skin, muscles and tissue from the surface of the skin to the surgical site providing an unobstructed pathway for the surgeon to work. Typically, a sequential. dilation technique is used to insert an access device. To minimize the damage to the tissue and muscles in creating a pathway, a small incision is made in the skin and a guide wire is inserted. Next a small diameter tubular member is advanced over the guide wire until it reaches the desired surgical site. As the tube is advanced it pushes the skin and tissue out of the way creating the surgical path. A second tubular member having a slightly larger diameter is then advanced over the first tubular member creating a wider path. These steps are repeated using tubular members of increasing diameter until the desired size of the path is created. Finally, an access device is advanced over the largest tubular member and the tubular members are removed leaving the access device in place. Using this technique minimizes trauma to the tissue as the tissue is expanded or stretched rather than cut as in an open procedure.

Some drawbacks with the dilator system include the number of steps it takes to create the desired size for the access device, and difficulty in inserting and removing the tubular members.

### SUMMARY

Disclosed herein are tissue expander systems and methods of use. In one embodiment a tissue expander system may include a tissue expander having a plurality of arms adapted to bear against tissue, the plurality of arms coupled at one end to an actuating member and at a second end to a shaft of the tissue expander, movement of the actuating member relative to the shaft adjusts the arms between a first position having a first diameter to a second position having a second diameter greater than the first diameter; and an outer sleeve having a lumen sized and shaped to receive the tissue expander in the second position.

Also disclosed herein is a tissue expander system kit including a plurality of tissue expanders having different diameters and a plurality of outer sleeves having different diameters corresponding to the diameters of the tissue expanders.

A method of creating a minimally invasive pathway to a vertebral body is also disclosed. The tissue expander having a plurality of arms shaped and sized to bear against tissue, the plurality of arms coupled at one end to an actuating member and to a shaft at a second end, is inserted in a first position through an incision into proximity with the vertebral body. The arms are adjusted from a first position to a second position by movement of the actuating member and an access device is placed over the tissue expander.

### BRIEF DESCRIPTION OF THE FIGURES

These and other features and advantages of the tissue expander system and methods disclosed herein will be more fully understood by reference to the following detailed description in conjunction with the attached drawings in which like reference numerals refer to like elements through the different views. The drawings illustrate principles of the tissue expander system and methods disclosed herein and, although not to scale, show relative dimensions.

FIG. 1A is a perspective view of the tissue expander in an unexpanded configuration;

FIG. 1B is a perspective view of the tissue expander shown in FIG. 1A in an expanded configuration;

FIG. 2A is a perspective view of the shaft of the tissue expander shown in FIG. 1A;

FIG. 2B is an alternate embodiment of a shaft of the tissue expander;

FIG. 3A is a perspective view of the arm of the tissue expander shown in FIG. 1A;

FIG. 3B is an end view of the arm shown in FIG. 3A;

FIG. 3C is a front view of the arm shown in FIG. 3A;

FIG. 4 is a perspective view of the linkage element of the tissue expander shown in FIG. 1A;

FIG. 5 is a perspective view of the actuating member of the tissue expander shown in FIG. 1A;

FIG. 6 is a perspective view of the outer sleeve, which in one embodiment may be used with the tissue expander of FIG. 1A creating an assembly;

FIG. 7A is a perspective view of the assembly of the tissue expander and the outer sleeve shown in FIGS. 1A and 6;

FIG. 7B is an end view of the assembly shown in FIG. 7A;

FIG. 8 is an exploded view of an assembly of the tissue expander of FIG. 1A and an access device;

FIG. 9 is an exploded view of an assembly of the tissue expander of FIG. 1A, the outer sleeve of FIG. 6, and an access device;

FIG. 10A is a perspective view of an alternate embodiment of a tissue expander;

FIG. 10B is a close-up view of the distal end of the tissue expander of FIG. 10A; and

FIG. 10C is a close-up view of the proximal end of the tissue expander of FIG. 10A.

### DETAIL DESCRIPTION OF EXEMPLARY EMBODIMENTS

Certain exemplary embodiments will now be described to provide an overall understanding of the principles of the structure, function, manufacture, and use of the tissue expander system and methods disclosed herein. One or more examples of these embodiments are illustrated in the accompanying drawings. Those of ordinary skill in the art will understand that the tissue expander system and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

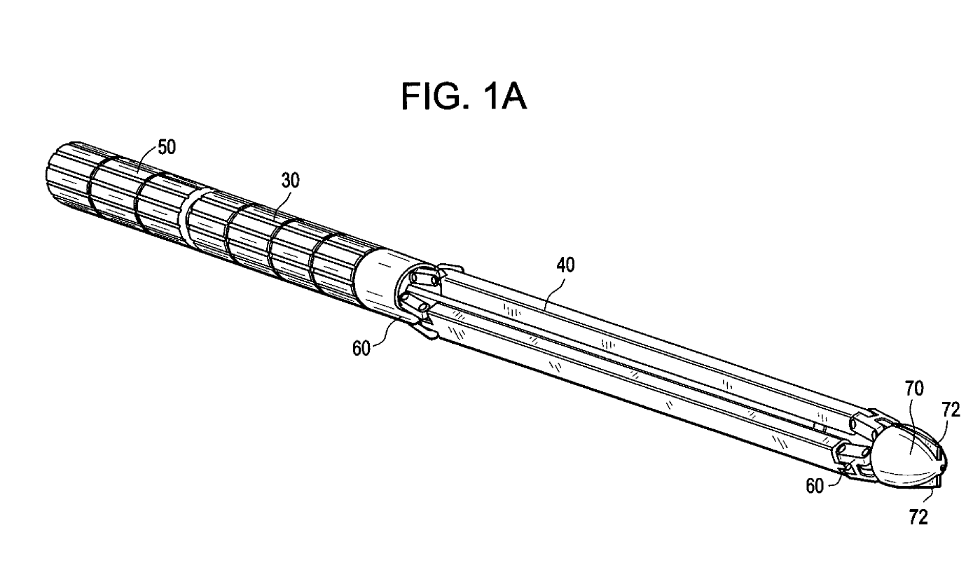

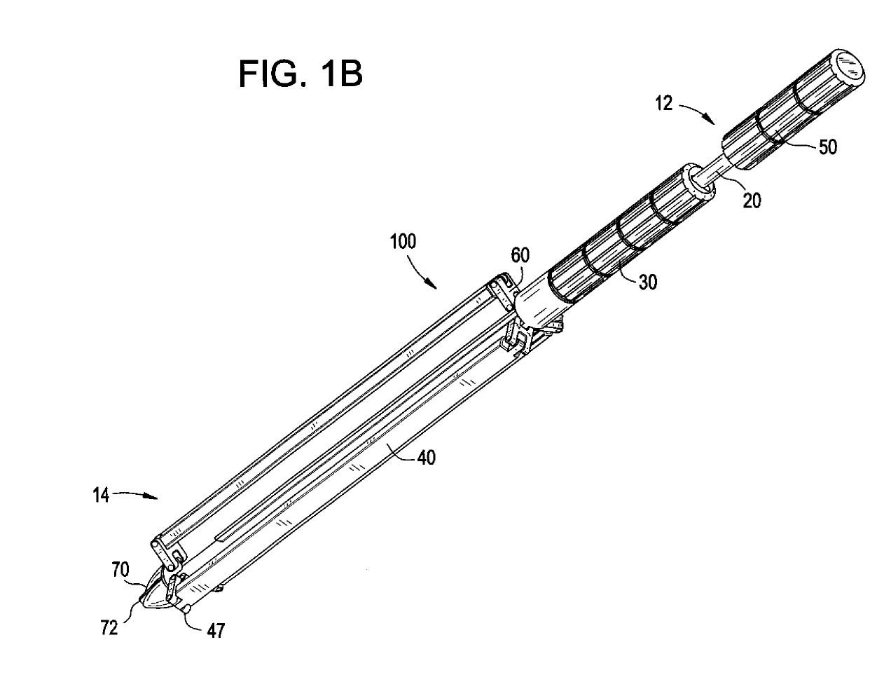

FIGS. 1-9 illustrate various components of an exemplary embodiment of a tissue expander system. The system may be used for numerous surgical procedures but will be described below in relation to spinal surgery. One skilled in the art will understand that the system can also be used for other surgical procedures including orthopedic procedures. The system may be used with any number of surgical approaches used in spinal surgery including anterior, posterior, anterior/lateral, lateral, and posterior/lateral.

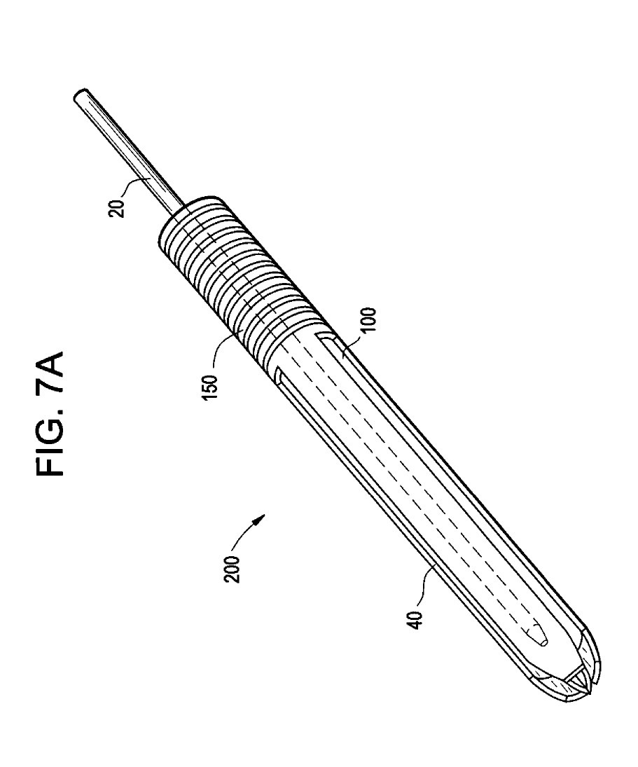

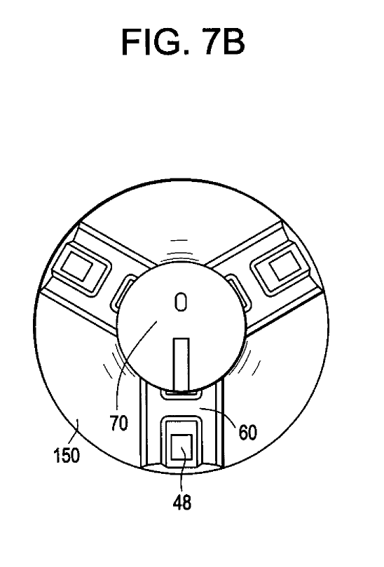

The exemplary tissue expander assembly 200 shown in FIG. 7A includes a tissue expander 100 and an outer sleeve 150. The exemplary tissue expander assembly 200 may be employed to expand skin, tissue, and muscle through a minimally invasive incision to create a pathway to a surgical site, i.e., a vertebral body. Various surgical approaches may be taken to the surgical site, including anterior, posterior or lateral. For example, the tissue expander 100 is inserted through a skin incision in a first position and advanced proximate to the vertebral body. The tissue expander 100 maybe adjusted to a second position to expand or stretch the surrounding tissue, muscle and skin. In one embodiment, the outer sleeve 150 may be advanced over the tissue expander 100 to form a cylindrical shaped assembly within the incision as shown in FIG. 7A and creating a pathway to the surgical site. An access device 500, such as a port or expandable retractor, may be advanced over the assembly to maintain the pathway during the surgical procedure. The tissue expander assembly 200 may be removed in one step, i.e., the tissue expander 100 and the outer sleeve 150 may be removed simultaneously, leaving the access device in place to define the pathway to the surgical site. The assembly 200, when employed in the exemplary manner, thus may be used to expand or dilate tissue, muscle or skin to create a pathway to the surgical site.

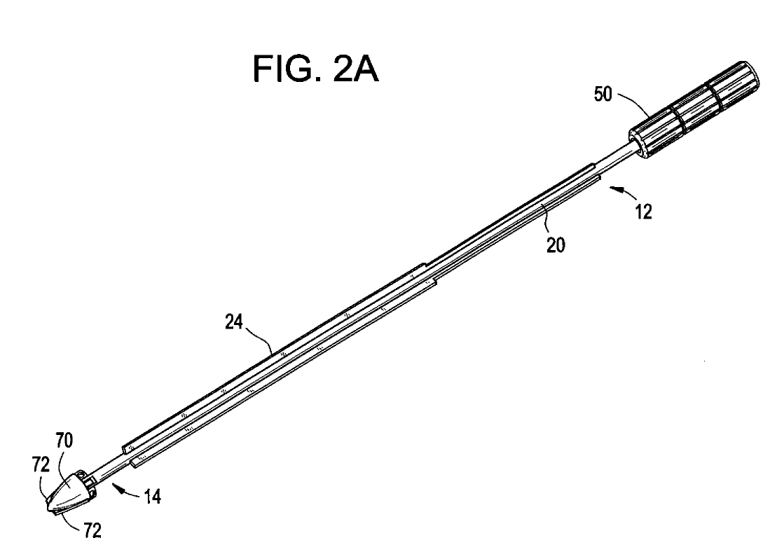

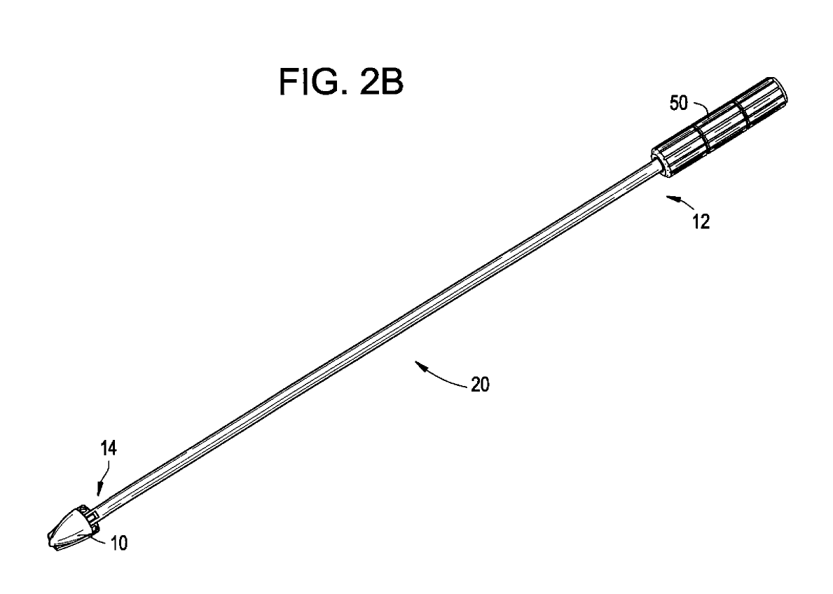

The tissue expander 100 of the exemplary assembly 200 illustrated in FIGS. 1A and 1B includes a shaft 20, an actuating member 30, a plurality of arms 40, a plurality of linkage members 60 and a handle 50. The shaft 20 extends from a proximal end 12 to a distal end 14, along a longitudinal axis and has a generally cylindrical shape as seen in FIGS. 2A-B. In one embodiment, the shaft 20 may have a plurality of ribs 24 radially spaced apart along a portion of the longitudinal axis of the shaft 20. The ribs 24 may provide additional bending strength to the shaft 20. Handle 50 may be positioned at the proximal end 12 of the shaft 20 as shown in FIG. 1B. The distal end 14 of the shaft 20 forms tip 70 which may have additional features for docking or bone preparation. For example, blunt edged blades 72 may extend from the tip 70 for preparing the bony surface to aid in docking of the tissue expander 100. The blades 72 may be shaped for scraping tissue off the bony surface. Alternately, the tip 70 may have serrated edges, teeth, or roughened surface area for better gripping of the bony surface. The tip 70 may have a blunt conical shape for ease of insertion through soft tissue.

The tissue expander 100 may have any number of arms 40. For example, in one exemplary embodiment, the tissue expander may have two arms. In another exemplary embodiment, the tissue expander may have six arms. In the exemplary embodiment, the tissue expander 100 has three arms, radially spaced 120 degrees from each other. One skilled in the art will appreciate that any number of arms could be used.

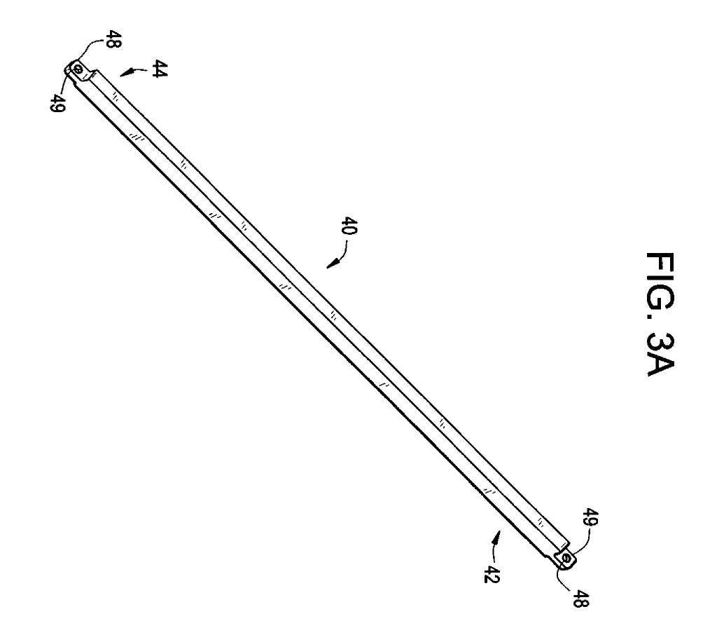

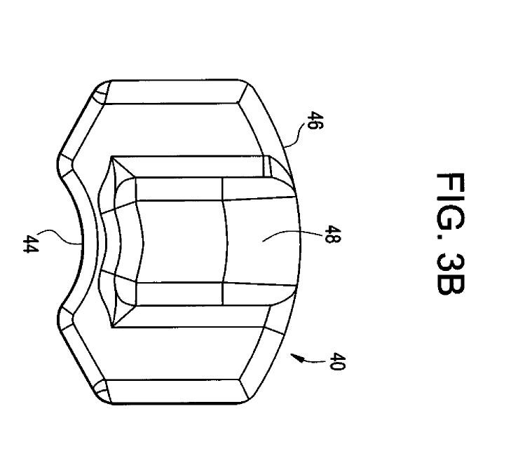

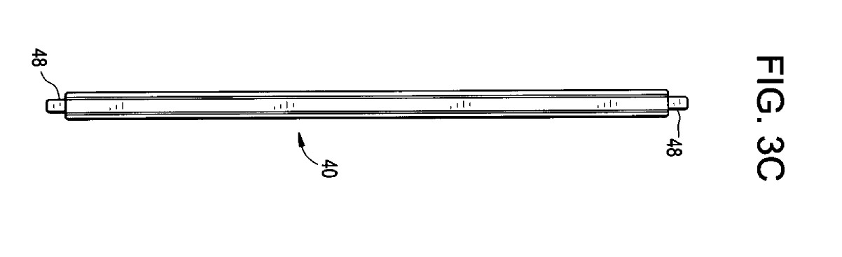

Arms 40 illustrated in FIGS. 3A-C, extend along a longitudinal axis from a proximal end 42 to a distal end 44. The arms 40 are sized to extend from the surgical site, e.g. proximate the patient's spine, to a point above the patient's skin. Arms 40 have an inner surface 44 and outer surface 46. The inner surface 44 is shaped to complement the shape of the shaft 20. In one embodiment, the shape of the inner surface 44 may be radiused to match the radius of the shaft 20. The outer surface 46 is sized and shaped to bear against tissue. In one embodiment, the outer surface 46 may have a generally arcuate shape. When the tissue expander 100 is in a first position, the inner surface 44 of the arms 40 fits flush against the shaft 20 and the outer surface 46 of the arms 40 align to give the tissue expander 100 a generally cylindrical shape having a first diameter. If the shaft 20 has ribs 24, the arms 40 are sized and shaped to nest between the ribs 24 in the first position. In one embodiment, the first diameter may be between 3 mm and 20 mm, in the exemplary embodiment, the first diameter may be between 5 mm and 10 mm. One skilled in the art will appreciate that the inner surface of the arms and the shaft may have other complementary shapes.

Arms 40 are coupled by linkage members 60 at the proximal end 42 to the actuating member 30 and at the distal end 44 to the shaft 20. The linkage members 60 form pivoting joints between the proximal ends of the arm 40 and the actuating member 30; and between the distal ends of the arms 40 and the distal end of the shaft 20. Movement of the actuating member 30 relative to the shaft 20, engages the linkage members 60 providing for movement of the arms 40 from a first position having a first diameter to a second position having a second diameter greater than the first diameter. In the second position, the arms 40 are spaced apart from one another. Arms 40 have tabs 48 extending from the proximal end 42 and the distal end 44 for connecting to the linkage members 60. Tabs 48 have a through hole 49 for receiving a linkage pin 47 to pivotally connect the arms 40 with the linkage members 60.

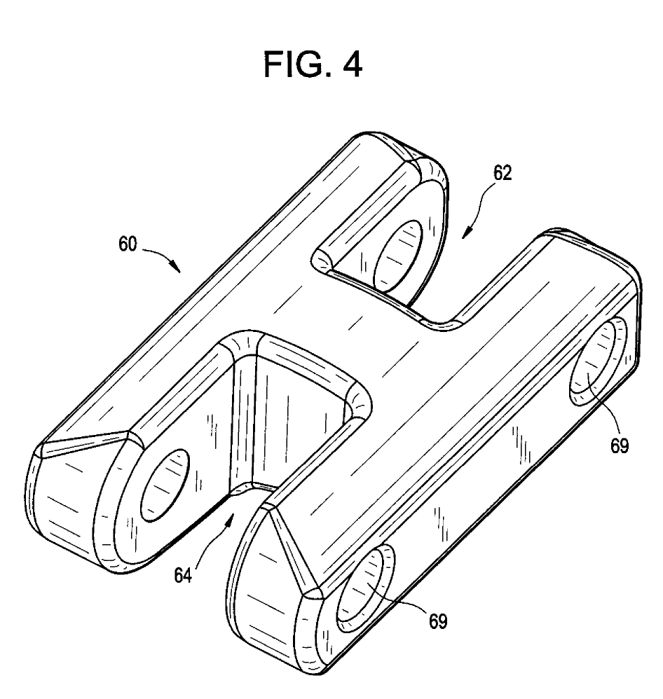

The linkage member 60 illustrated in FIG. 4 has a generally H-shaped body. The linkage member 60 has a first opening 62 and a second opening 64. Tab 48 of arm 40 is sized and shaped to fit in either the first opening 62 or the second opening 64. Linkage member 60 has through holes 69 for receiving a linkage pin 47 to pivotally connect the arms 40, the actuating member 30 or the shaft 20 with the linkage member 60. The length of the linkage member 60 determines the distance that the arms 40 will expand radially from their first position. The length of the linkage member 60 corresponds to the second diameter of the tissue expander 100 in its second position.

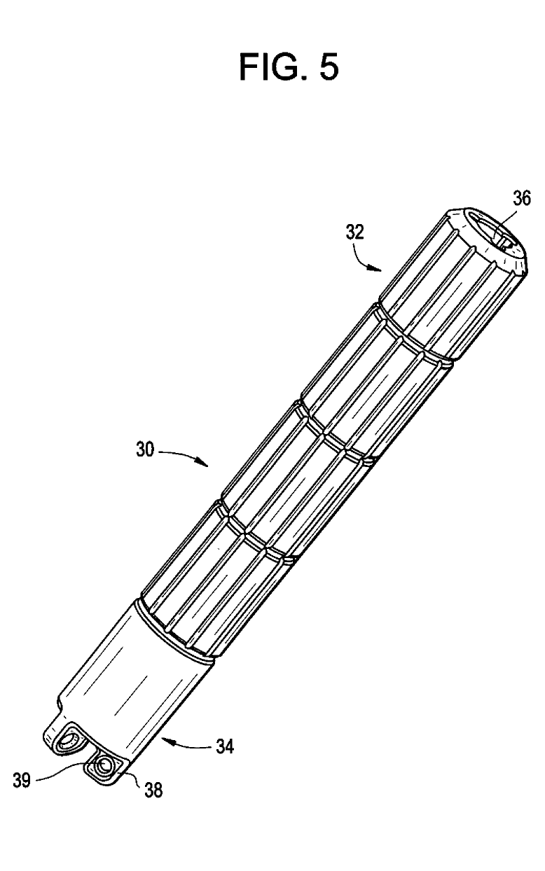

One embodiment of the actuating member 30, illustrated in FIG. 5, has a generally cylindrical shape with a central lumen 36 extending along a longitudinal axis from a proximal end 32 to a distal end 34. The lumen 36 is sized and shaped to receive the shaft 20 of the tissue expander 100. The shaft 20 may extend through the distal end 34 and proximal end 32 of the actuating member 30. The shaft 20 may be slidably received within the actuating member 30. The distal end 34 of the actuating member 30 may have tabs 38 adapted to couple the actuating member 30 with linkage member 60 and arms 40. The tabs 38 may be sized and shaped to fit within either the first opening 62 or the second opening 64 of the linkage member 60 and have a through hole 39 adapted to receive a linkage pin for coupling the linkage member 60 to the actuating member 30. The outer surface of the actuating member 30 may be textured for improved gripping. The actuating member 30 may translate axially along the longitudinal axis of the shaft 20. In alternate embodiments, the actuating member may have a threaded mechanism, a ratcheting mechanism or any other mechanism that provides a mechanical advantage to move the arms from a first position to a second position relative to the shaft.

The tissue expander 100 is inserted through a minimally invasive incision in the first position such that the arms 40 sit flush against the shaft 20 and the linkage members 60 are aligned parallel to the longitudinal axis of the shaft 20. In this position the actuating member 30 abuts the handle 50 at the proximal end of the shaft 20. The tip 70 of the tissue expander 100 separates soft tissue as it advances through the incision to the desired surgical site. The tissue expander 100 may be wanded (e.g., moved repeatedly in an anterior-posterior, cephalad-caudal, or medial-lateral orientation) to further separate soft tissue near the surgical site. The actuating member 30 may be moved toward the distal end 14 of the shaft 20. As the actuating member 30 is engaged, the linkage members 60 adjust the arms 40 from a first position to a second position in a radial direction. The actuating member 30 stops movement when the linkage members 60 are perpendicular to the longitudinal axis of the shaft 20. The arms 40 in the second position are radially spaced apart from each other creating a second, larger, diameter of the tissue expander 100. The second diameter may be between 10 mm and 30 mm or in the exemplary embodiment, may be between 12 mm and 24 mm. An outer sleeve 150, configured to have the same diameter as the tissue expander 100 in the second position, may be advanced over the tissue expander.

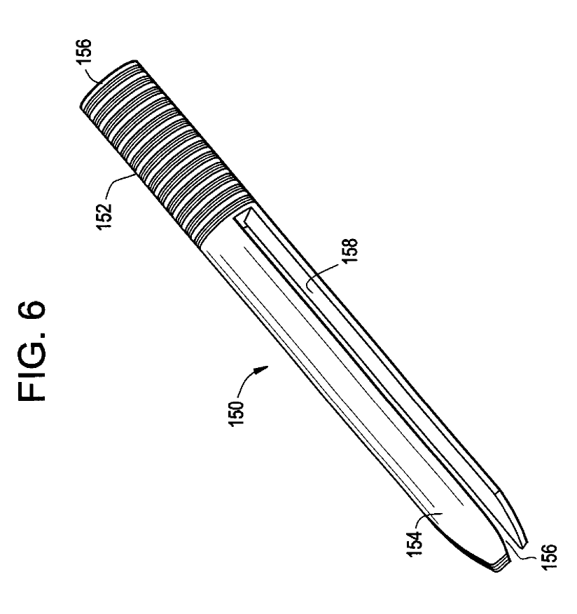

The outer sleeve 150 of the tissue expander assembly 100 shown in FIG. 6 has a generally tubular shape with a central lumen 156 extending from a distal end 154 to a proximal end 152. The outer sleeve 150 is sized and shaped to receive the tissue expander 100 in the second position. Extending proximally from the distal end 154 along the longitudinal axis of the outer sleeve 150 are slots 158. The number of slots 158 corresponds to the number of arms 40 on the tissue expander 100. As illustrated in FIG. 7A, each slot 158 is sized and shaped to receive a portion of the arm 40 of the tissue expander 100 in the second position. The length of each slot 158 is adapted to accommodate the length of the arm 40. The distal end 154 of the outer sleeve 150 may be tapered to ease insertion. The length of the outer sleeve 150 is less than the overall length of the tissue expander 100, such that the proximal end of the shaft 20 extends through the lumen 156 of the outer sleeve 150. The outer sleeve 150 may have a generally circular or ellipsoidal shape depending on the shape of the access device 500 to be used. The outer diameter of the outer sleeve 150 is the same as or greater than the second diameter of the tissue expander 100 in the expanded position. The slots 158 of the outer sleeve 150 are sized to receive the arms 40 such that the outer sleeve 150 and the arms 40 in the second position cooperate to form a cylinder having a continuous outer surface along at least a portion of the length of the outer sleeve 150, as illustrated in FIGS. 7A and 7B.

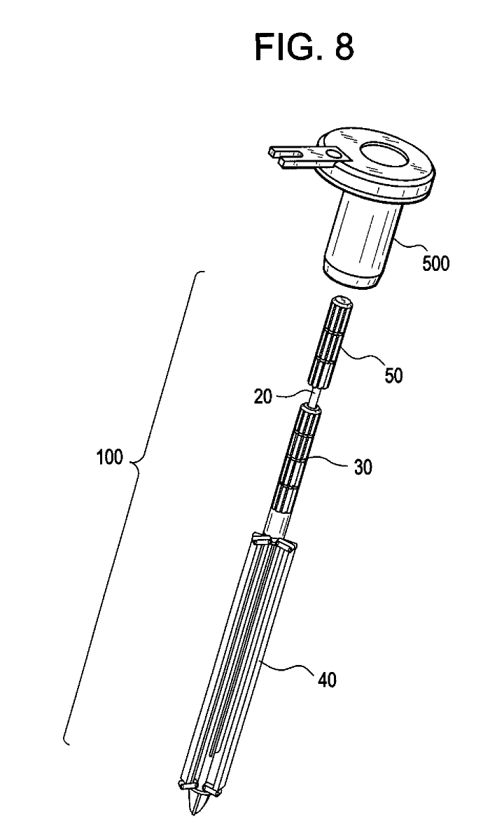

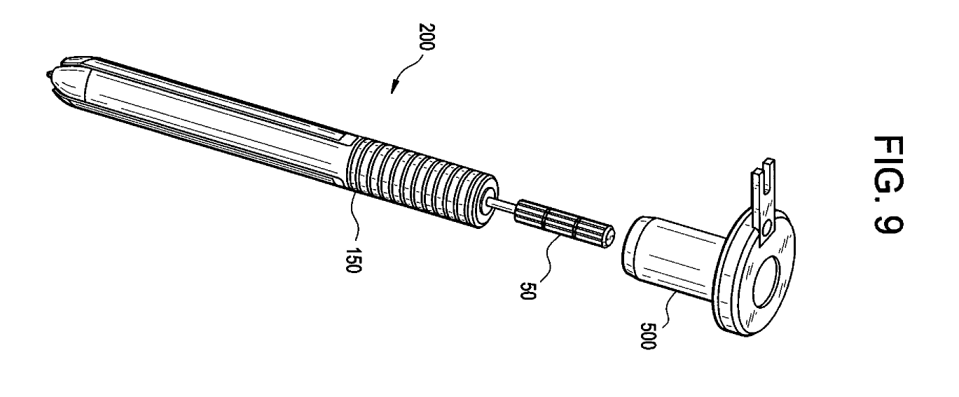

The outer sleeve 150 is advanced through the incision over the tissue expander 100. The slots 158 receive the arms 40 as shown in FIG. 7A. The arms 40 and the outer sleeve 150 cooperate to form a tissue expander assembly 200 having a continuous outer surface along at least a portion of the length of the outer sleeve 150 that retracts tissue and muscles along the pathway to the surgical site. FIG. 7B illustrates the continuous outer surface of the assembly with an end view of the assembly 200. An exemplary embodiment of the access device 500 shown in FIG. 9 may be placed over the tissue expander assembly 200 to create the pathway to the surgical site. The tissue expander assembly 200 may be removed in one step by withdrawing the assembly by the handle 50, leaving the access device 500 in place. Alternately, as shown in FIG. 8, the access device 500 may be placed directly over the tissue expander 100 without using the outer sleeve 150.

A kit may be provided including a plurality of tissue expanders 100 and the outer sleeves 150 in varying sizes (diameters and lengths) correlating to the various sizes of the access devices 500 available for use. The length of the linkage member 60 changes according to the desired diameter size of the access device 500. The arms 40 and/or actuating member 30 may have depth markings for measuring the depth at the skin incision for assistance in determining the length of the access device 500 to be used. The tissue expander 100 and outer sleeve 150 may be manufactured from any biocompatible material such as metal, plastic, or composite and may be radiopaque or radiolucent. If radiolucent, the arms 40, actuating member 30 and/or outer sleeve 150 may also include depth markers made from radiopaque rings for intra-operative depth measurements under fluoroscopy. Alternately, the distal ends of the tissue expander 100 and outer sleeve 150 may be radiopaque to aid the surgeon under fluoroscopy.

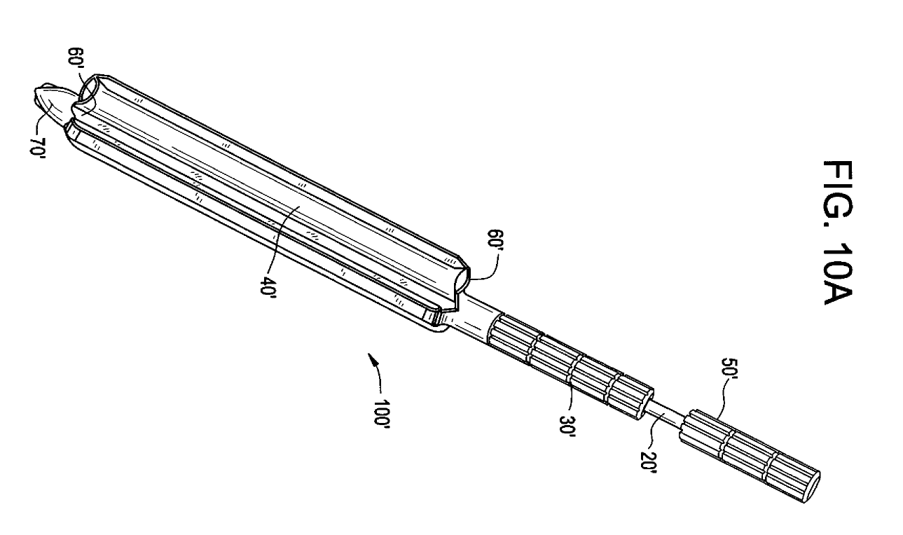

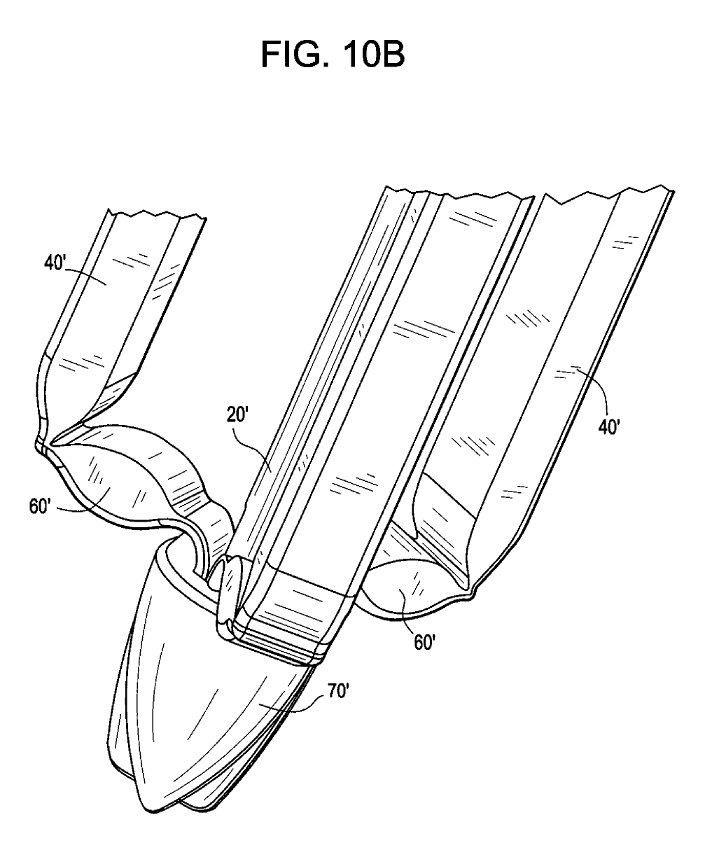

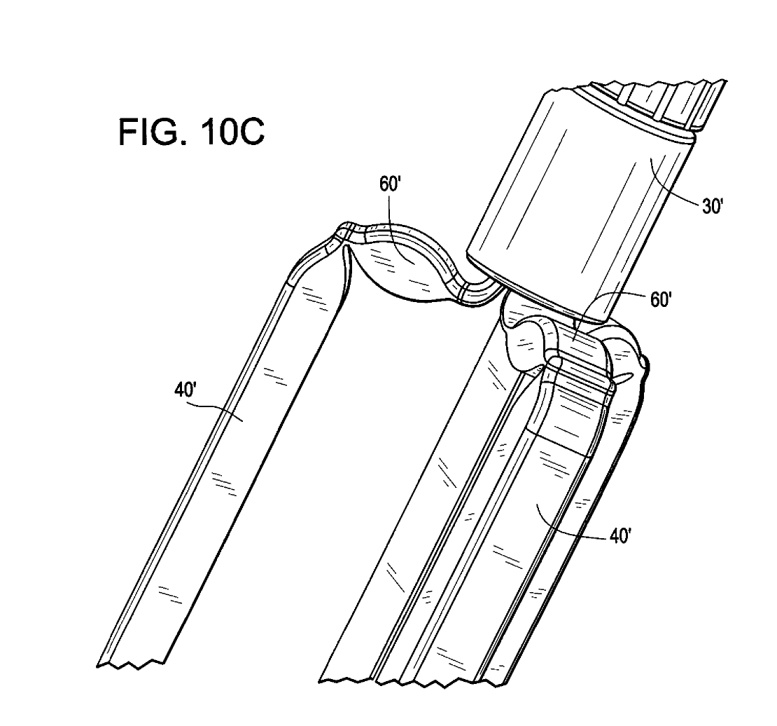

An alternate embodiment of a tissue expander 100′ illustrated in FIGS. 10A-C may include a shaft 20′ coupled to a unitary piece including an actuating member 30′, a tip 70′, and arms 40′. The actuating member 30′ may be coupled to arms 40′ at the proximal end by a living hinge 60′. The arms 40′ may be coupled at the distal end to the tip 70′ by a living hinge 60′. The shaft 20′ may be connected to the unitary piece at the tip 70′ by threads, snap-fit or ultrasonic weld. The unitary piece may be made of super-elastic material such as plastic or nitinol. The shaft 20′ may be made of metal, a composite material, or overmolded. The living hinge 60′ is formed from thinned portions of the unitary piece which flex upon movement by the actuating member 30′ allowing for movement of the arms from a first position to a second position relative to the shaft 20′.

While the tissue expander systems and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

1. A tissue expander system comprising:
a tissue expander having a plurality of arms adapted to bear against tissue, the plurality of arms coupled at one end to an actuating member and at a second end to a a shaft of the tissue expander, movement of the actuating member relative to the shaft adjusts the arms between a first position having a first diameter to a second position having a second diameter greater than the first diameter; and
an outer sleeve having a lumen sized and shaped to receive the tissue expander in the second position.

2. The tissue expander system of claim 1 wherein the arms are spaced apart from one another in the second position.

3. The tissue expander system of claim 2 wherein the outer sleeve includes a plurality of longitudinal slots, the arms being positionable in the slots when the arms are in the second position.

4. The tissue expander system of claim 3 wherein the outer sleeve and the arms in the second position cooperate to form a cylinder having a continuous outer surface along at least a portion of the length of the outer sleeve.

5. The tissue expander system of claim 1 wherein the actuating member is slidable relative to the shaft.

6. The tissue expander system of claim 1, wherein the actuating member has a ratcheting mechanism.

7. The tissue expander system of claim 1, wherein the actuating member has a threaded mechanism.

8. The tissue expander system of claim 1, wherein the shaft has tip for docking to bone at the surgical site.

9. The tissue expander system of claim 8 wherein the tip has blades for scraping tissue off bone.

10. The tissue expander system of claim 1 wherein the arms are coupled to the actuating member and shaft by linkage members forming a pivoting joint.

11. The tissue expander system of claim 10 wherein the linkage member is a living hinge.

12. The tissue expander system of claim 1 further comprising an access device for positioning over the tissue expander and the outer sleeve.

13. The tissue expander system of claim 12 wherein the access device is a port.

14. The tissue expander system of claim 12 wherein the access device is an expandable retractor.

15. The tissue expander system of claim 1 wherein the inner surface of the arm is shaped to complement the shape of the shaft.

16. The tissue expander system of claim 1, further comprising a handle on the proximal end of the shaft.

17. A tissue expander system kit for expanding tissue at a surgical site, comprising:
a plurality of tissue expanders having different diameters, and
a plurality of outer sleeves having different diameters corresponding to the diameters of the plurality of tissue expanders.

18. A method of creating a minimally invasive pathway to a vertebral body comprising:
inserting a tissue expander having a plurality of arms shaped and sized to bear against tissue, the plurality of arms coupled at one end to an actuating member and to a shaft at a second end, in a first position through an incision into proximity with the vertebral body;
adjusting the arms from a first position to a second position by movement of the actuating member; and
placing an access device over the tissue expander.

19. The method of claim 18 further comprising creating an incision for an anterior surgical approach to the vertebral body.

20. The method of claim 18 further comprising creating an incision for a posterior surgical approach to the vertebral body.

21. The method of claim 18 further comprising creating an incision for a lateral surgical approach to the vertebral body.

22. The method of claim 18 further comprising inserting an outer sleeve over the tissue expander into proximity with the vertebral body.

23. The method of claim 22 further comprising:
simultaneously removing the tissue expander and the outer sleeve.
